(19) United States
(12) Patent Application Publication
(10) Pub. No.: US 2010/0204449 A1
(43) Pub. Date: Aug. 12, 2010

(54) METHODS AND INTERMEDIATES FOR CHEMICAL SYNTHESIS OF POLYPEPTIDES AND PROTEINS

(76) Inventors: Zheng Xin Dong, Holliston, MA (US); Sun H. Kim, Needham, MA (US)

Correspondence Address: Alan F. Feeny, Esq., Biomeasure, Incorporated, 27 Maple Street, Milford, MA, 01757-3650, US

(21) Appl. No.: 12/676,332

(22) PCT Filed: Aug. 28, 2008
(86) PCT No.: PCT/US08/10226

§ 371 Date: Mar. 3, 2010

Related U.S. Application Data
(60) Provisional application No. 60/967,303, filed on Sep. 4, 2007.

(51) Int. Cl. C07K 1/107; C07K 14/00; C07K 2/00; C07C 229/02

(52) U.S. Cl. 530/345; 530/402; 530/300; 562/556

(57) ABSTRACT

The present invention relates to methods and intermediates for chemical synthesis of polypeptides and proteins, and more particularly to methods and intermediates for chemically ligating a peptide fragment containing N-terminal β-methyl-cysteine (SEQ ID NO: 1) with another peptide fragment having C-terminal thioester to generate a β-amino-thioester intermediate that spontaneously rearranges to form an amide bond. The invention also relates to methods of synthesizing β-methyl-cysteine (SEQ ID NO: 1) and its protected forms. Furthermore, the invention relates to converting a β-methyl-thiazolidine residue to a β-methyl-cysteine (SEQ ID NO: 1) residue of polypeptides and proteins.

### BACKGROUND OF THE INVENTION

The present invention relates to methods and intermediates for chemical synthesis of polypeptides and proteins, and more particularly to methods and intermediates for chemically ligating a peptide fragment containing N-terminal beta-methyl-cysteine (“β-methyl-cysteine”; (SEQ ID NO:1)) with another peptide fragment having C-terminal thioester to generate a beta-amino-thioester (“β-amino-thioester”) intermediate that spontaneously rearranges to form an amide bond. Furthermore, the invention relates to methods of synthesizing β-methyl-cysteine and beta-methyl-thiazolidine (“β-methyl-thiazolidine”; (SEQ ID NO:1)).

Several techniques for chemically synthesizing proteins have been developed. See, e.g., Stewart, J. M. et al., Solid Phase Peptide Synthesis (Pierce Chemical Co., 2d ed., 1984), and Bodanszky, M. et al., The Practice of Peptide Synthesis (Springer-Verlag, 1984). Among them, native chemical ligation has proven to be one of most useful methods for chemically generating native proteins. However, native chemical ligation is only suitable for the synthesis of polypeptides and proteins having cysteine residue(s), which can be used as the connection point(s) for ligating peptide fragments to form the final target polypeptides and proteins.

To produce polypeptide and protein analogs and derivatives which have improved chemical and biological properties, incorporation of unnatural amino acid residue(s) into specific position(s) inside the polypeptides and proteins is sometimes required. Such analogs and derivatives can have improved chemical stability, improved enzymatic stability, prolonged duration of action in vivo, and enhanced biological activities. One class of such unnatural amino acids is the β-methyl-cysteine (SEQ ID NO:1). The β-methyl-cysteine (SEQ ID NO:1) can impose conformational constraint on disulfide bridge and peptide backbone and potentially protect the peptide bonds against enzymatic cleavage (see, e.g., Haviv, F. et al., J. Med. Chem., 1993, 36:363-369; Failie, D. P., et al., Curr. Med. Chem., 1995, 2:654-686; Miller, S. M., et al., Drug Dev. Res., 1995, 35:20-32; and Schmidt, R., et al., Int. J. Pept. Protein Res., 1995, 46:47-55). To generate biologically more active, enzymatically more stable protein analogs and derivatives, there is a need to develop a new chemical method to incorporate β-methyl-cysteine (SEQ ID NO:1) residue in any desired position inside of proteins.

### BRIEF DESCRIPTION OF THE DRAWINGS

FIG. 1 is an illustration showing a synthetic scheme for forming an amide bond in accordance with one aspect of the present invention.

FIG. 2 is an illustration showing a synthetic scheme for protecting N-terminal β-methyl-cysteine (SEQ ID NO:1) in a β-methyl-thiazolidine form during the chemical synthesis of polypeptides and proteins in accordance with one aspect of the present invention.

FIG. 3 is an illustration showing a synthetic scheme for synthesizing N-Boc-S-trityl-β-methyl-cysteine (SEQ ID NO:12) and N-Fmoc-S-trityl-β-methyl-cysteine (SEQ ID NO:3).

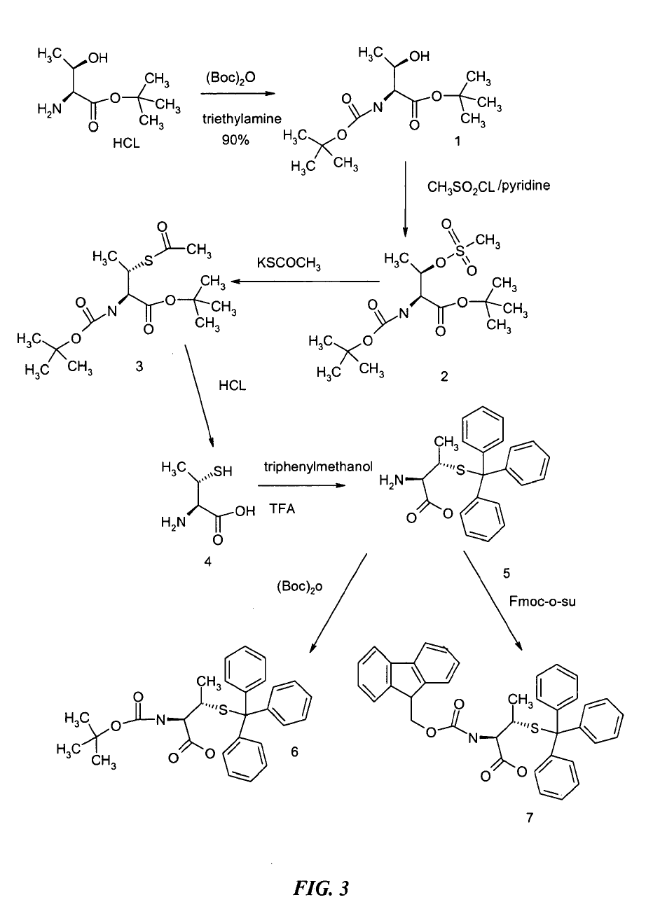

### SUMMARY OF THE INVENTION

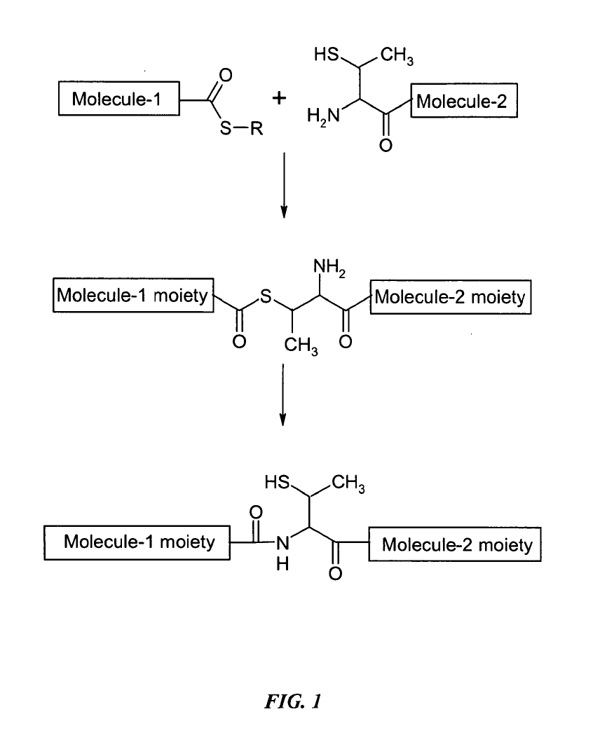

The present invention is directed to a method of forming an amide bond between two molecules that can be proteins, polypeptides, peptidomimetics, polymers, or any combination thereof. Between these two molecules, one—the “first”—molecule contains a terminal β-methyl-cysteine (SEQ ID NO:1) residue and the other—the “second”—molecule contains a thioester functional group. During the reaction, the two molecules first connect through a thioester linkage, and then, the β-amino-thioester linkage spontaneously converts to the final amide bond via an intramolecular rearrangement, as shown in FIG. 1. The resulting final product contains the moieties of the two molecules which are connected through the newly formed amide bond.

Another aspect of the present invention is directed to a ligation reaction which is carried out in a solution or solid phase. The reaction medium may contain thiol catalyst(s). Such thiol catalysts include, but are not limited to, thiophenol, 1-thio-2-nitrophenol, 2-thio-benzoic acid, 2-thio-pyridine, 4-thio-2-pyridinecarboxylic acid, 4-thio-2-nitropyridine, 4-mercaptophenylacetic acid, 2-mercaptoethanesulfonic acid, 3-mercapto-1-propanesulfonic acid, and 2,3-dimercaptopropanesulphonic acid.

Another aspect of the present invention is directed to the conversion of β-methyl-thiazolidine to β-methyl-cysteine (SEQ ID NO:1). For this stepwise ligation, a peptide fragment bearing N-terminal β-methyl-cysteine (SEQ ID NO:1) and C-terminal thioester is needed. However, the conventional thio-protecting groups, such as benzyl for Boc-chemistry and trityl and t-butyl for Fmoc-chemistry, cannot be used to protect the sulfhydryl group of the N-terminal (3-methyl-cysteine (SEQ ID NO:1). This is due to the fact that during the final cleavage step, such a conventional protecting group will be removed and a free sulfhydryl group will be generated which will react with the C-terminal thioester to form undesired products.

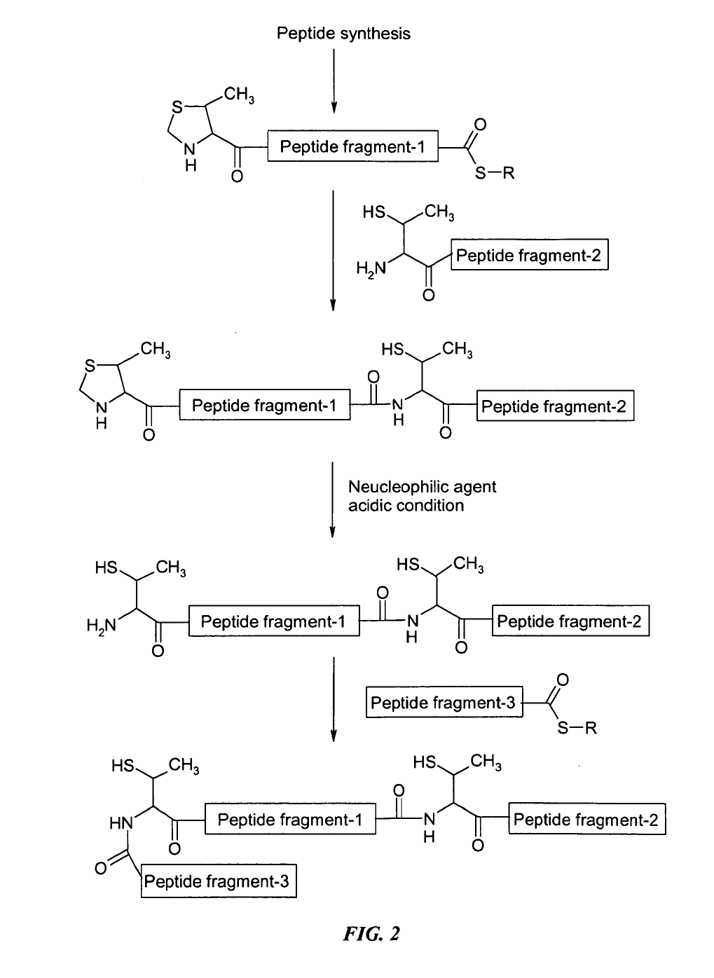

To address this problem, the present invention provides a method for protecting N-terminal β-methyl-cysteine (SEQ ID NO:1) in a β-methyl-thiazolidine form during the chemical synthesis of polypeptides and proteins, as shown in FIG. 2. The β-methyl-thiazolidine will stay intact during the cleavage step and a peptide intermediate containing N-terminal β-methyl-thiazolidine and C-terminal thioester will be generated. This intermediate will be used in the ligation reaction with another peptide fragment containing C-terminal thioester. Only after the ligation reaction, the β-methyl-thiazolidine in the product will be converted to a free β-methyl-cysteine (SEQ ID NO:1) by using a neucleophilic agent under acidic condition, wherein said nucleophilic agent is O-alkylhydroxylamine, and more specifically, wherein said O-alkylhydroxylamine is O-methylhydroxylamine, and wherein said acidic conditions are in the range of pH 2.0 to pH 6.0. The resulting bigger peptide fragment having N-terminal β-methyl-cysteine (SEQ ID NO:1) residue can be used for further ligation step to generate even bigger polypeptides or proteins.

### DETAILED DESCRIPTION OF THE INVENTION

Certain amino acids present in compounds of the invention can be and are represented herein as follows:
Ala or A is alanine,
Arg or R is arginine,
Asn or N is asparagine,
Asp or D is aspartic acid,
Cys or C is cysteine,
Gln or Q is glutamine,
Glu or E is glutamic acid,
Gly or G is glycine,
His or H is histidine,
Ile or I is isoleucine,
Leu or L is leucine,
Lys or K is lysine,
Met or M is methionine,
Nle is norleucine,
Phe or F is phenylalanine,
Pro or P is proline,
Ser or S is serine,
Thr or T is threonine,
Trp or W is tryptophan,
Tyr or Y is tyrosine, and
Val or V is valine.

Certain other abbreviations used herein are defined as follows:
Boc is tert-butyloxycarbonyl,
Bzl is benzyl,
DCM is dichloromethane,
DIC is N,N-diisopropylcarbodiimide,
DIEA is diisopropylethyl amine,
Dmab is 4-{N-(1-(4,4-dimethyl-2,6-dioxocyclohexylidene)-3-methylbutyl)-amino}benzyl,
DMAP is 4-(dimethylamino)pyridine,
DMF is dimethylformamide,
DNP is 2,4-dinitrophenyl,
Fmoc is Fluorenylmethyloxycarbonyl,
HBTU is 2-(1H-benzotriazole-1-yl)-1,1,3,3-tetramethyluronium hexafluorophosphate,
cHex is cyclohexyl,
HOAt is O-(7-azabenzotriazol-1-yl)-1,1,3,3-tetramethyluronium hexafluorophosphate,
HOBt is 1-hydroxy-benzotriazole,
Mmt is 4-methoxytrityl,
NM is N-methylpyrrolidone,
Pbf is 2,2,4,6,7-pentamethyldihydrobenzofuran-5-sulfonyl,
Ph is phenyl,
tBu or t-butyl is tert-butyl,
TIS is triisopropylsilane,
TOS is tosyl,
Trt is trityl,
TFA is trifluoro acetic acid,
TFFH is tetramethylfluoroforamidinium hexafluorophosphate, and
Z is benzyloxycarbonyl.

All abbreviations (e.g., Ala) of amino acids in this disclosure stand for the structure of —NH—C(R)(R′)—CO—, wherein R and R′ each is, independently, hydrogen or the side chain of an amino acid (e.g., R═CH₃ and R′═H, for Ala), or R and R′ may be joined to form a ring system.

What is meant by “beta-methyl-cysteine”, “β-Me-Cys” or “β-MeCys” (SEQ ID NO:1), which terms are equivalents of each other, is:

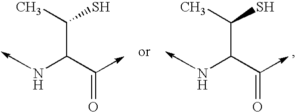

which can be in L- or D-configuration.

What is meant by “β-methyl-thiazolidine”, “βMe-Thz”, “β-MeThz” or “βMeThz”, which terms are equivalents of each other, is:

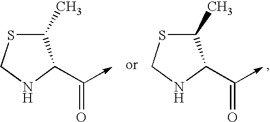

which can be in L- or D-configuration.

What is meant by “β-amino-thioester” or “β-(amino)-thioester”, which terms are equivalents of each other, is:

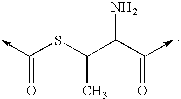

### Examples

Examples are provided below to further illustrate different features of the present invention. The examples also illustrate useful methodology for practicing the invention. These examples do not limit the claimed invention.

The peptide fragments used in this invention can be prepared by standard solid phase peptide synthesis (See, e.g., Stewart, J. M. et al., Solid Phase Peptide Synthesis (Pierce Chemical Co., 2d ed. 1984)).

### Example 1

### N-Boc-threonine-t-butylester (SEQ ID NO:4)

To an ice cooled suspension of threonine-t-butylester•HCl (2.2 g, 10 mmoles; (SEQ ID NO:5)) in 30 ml of dichloromethane) was added 4.2 ml of triethylamine (3 equivalents) followed by a solution of di-tert-butyl-dicarbonate ((Boc)₂O, 2.8 g, 1.2 equivalents) in 2 ml of dichloromethane and the mixture was slowly allowed to reach room temperature. After stirring for 2 hours, the mixture was diluted with 20 ml of chloroform and the mixture was washed with water and dried (MgSO₄). Volatile substances were removed in vacuo to dryness. Viscous oil was obtained (2.8 g). It was used directly in the next reaction without further purification.

### Example 2

### N-Boc-O-methanesulfonyl-threonine-t-butylester (SEQ ID NO:6)

To a stirred solution of N-Boc-threonine-t-butylester (2.8 g; (SEQ ID NO:4)) in 40 ml of pyridine cooled to −20° C. was added dropwise 2 ml of methanesulfonylchloride (2.5 equivalents), and the mixture was allowed to reach room temperature. After stirring overnight, the reaction mixture was poured into ice-water (300 ml), and it was extracted with ether (2×250 ml). The ether extract was washed with 0.4N HCl until washing became acidic (pH 4) and dried (MgSO₄). Solvent was removed in vacuo to dryness. A pale solid was obtained (3.3 g). TLC (silica gel, chloroform/acetone=9:1, Rf=0.56, ninhydrin spray).

### Example 3

### Allo-N-Boc-S-acetyl-β-methyl-cysteine-t-butylester (SEQ ID NO:7)

To a solution of 2.5 ml of thiolacetic acid (30 mmloes) in 20 ml of methanol was added 24 ml of 1.0N KOH in methanol under nitrogen atmosphere and after stirring for 1 hour, volatile substances were removed in vacuo to give potassium thiolacetate, a pale yellow solid. It was dissolved in 30 ml of dimethylformide, cooled in an ice bath and a solution of N-Boc-O-methanesulfonyl-threonine-t-butylester (crude 3.3 g; (SEQ ID NO:6)) in 20 ml of dimethylformide was added dropwise under nitrogen atmosphere. The reaction mixture was allowed to room temperature under nitrogen atmosphere, stirred overnight (gel formation). Solvent was removed in vacuo to dryness. The residue was partitioned between ethyl acetate and water. Ethyl acetate layer was washed with water and dried (MgSO₄). After evaporation of solvent, the residue was chromatographed over silica gel (60 g) using chloroform/acetone=195:5 as eluents. Appropriate fractions were pooled and solvents were removed in vacuo to dryness. A brown viscous substance was obtained (2.3 g). TLC (silica gel, chloroform/ethyl acetate=9:1, Rf=0.82).

### Example 4

### Allo-β-methyl-cysteine (SEQ ID NO:8)

Allo-N-Boc-S-acetyl-β-methyl-cysteine-t-butylester (680 mg, 2 mmoles; (SEQ ID NO:7)) was treated with 10 ml of trifluoroacetic acid for 1 hour. After volatile substances were removed in vacuo to dryness, the residue was dissolved in 10 ml of 6N HCl and heated at 80˜85° C. (oil bath) overnight under nitrogen atmosphere. Excess HCl and water were removed in vacuo to dryness and the residue was lyophilized Electro-spray ionization mass spectrometry (ESI-MS) analysis showed 136.5 and other peaks. It was used in the next reaction directly without further purification. It was noted that replacement of nitrogen by argon and degassing of 6N HCl can improve the yield.

### Example 5

### Allo-S-trityl-β-methyl-cysteine (SEQ ID NO:9)

The mixture of crude allo-β-methylcysteine (2 mmole scale; (SEQ ID NO:10)) and triphenylmethanol (520 mg, 1 equivalent) was treated with 10 ml of trifluoroacetic acid for 30 minutes and the volatile substances were removed in vacuo to dryness. A pale yellow solid was obtained. ESI-MS gave 377.7.

### Example 6

### Allo-N-Boc-S-trityl-β-methyl-cysteine (SEQ ID NO:11)

Crude allo-S-trityl-β-methyl-cysteine (SEQ ID NO:9) was dissolved in 20 ml of acetonitrile and treated with aqueous NaHCO₃ to make the solution pH 8 and a solution of tert-butyl dicarbonate (520 mg) in 5 ml of acetonitrile was added. After 2 hours, the reaction mixture was cooled, acidified with 5% KHSO₄ and it was extracted with ethyl acetate (50 ml), and dried (MgSO₄). After evaporation of solvent, the residue was chromatographed on silica gel (50 g) using chloroform/methanol=195:5 as eluents. Appropriate fractions were pooled and solvents were removed in vacuo to dryness. A white foam was obtained (160 mg). TLC (silica gel, chloroform/methanol=4:1, Rf=0.51). ESI-MS gave 500.2 and 243.3 peaks.

### Example 7

### Allo-N-Fmoc-S-trityl-β-methyl-cysteine (SEQ ID NO:12)

The following is a description of preparation of allo-N-Fmoc-S-trityl-β-methyl-cysteine (SEQ ID NO:12). It was prepared in analogous manner similar to allo-N-Boc-S-trityl-β-methyl-cysteine (SEQ ID NO:11). Fmoc-osu was used instead of tert-butyl dicarbonte. TLC (silica gel, chloroform/methanol=9:1, Rf=0.27). ESI-MS gave 622.0.

### Example 8

### Preparation of Model Peptide β-MeCys-Lys-Phe-NH₂ (SEQ ID NO:13)

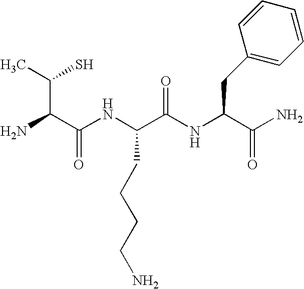

The title peptide was synthesized on a manual peptide synthesizer. Rink amide MBHA Resin (106 mg, 76 micromole, 0.72 mmole/g) (Novabiochem, San Diego, Calif., USA) was used. The Fmoc amino acids were used with the following side chain protection: Boc-β-MeCys(Trt)-OH (SEQ ID NO:14), Fmoc-Lys(Boc)-OH (Novabiochem, San Diego, Calif., USA; (SEQ ID NO:15)), and Fmoc-Phe-OH (Novabiochem, San Diego, Calif., USA; (SEQ ID NO:16)). The Fmoc groups were removed by treatment with 25% piperidine in DMF for 10 min and repeated for 20 minutes. In each coupling step, the Fmoc amino acid (4 equivalents), HOBt (4 equivalents) and DIC (4 equivalents) in NMP were used. The following reaction cycle was used: (1) washing with DMF; (2) removing Fmoc protecting group with 25% piperidine in DMF for 30 minutes; (3) washing with DMF; and (4) coupling with pre-activated Fmoc amino acid for 90 minutes. Boc-beta-MeCys(Trt)-OH (50.3 mg 0.105 mmole; (SEQ ID NO:14)) was coupled using TFFH (27.8 mg, 0.105 mmole), and DIEA (27 mg, 0.211 mmole) in NMP for 12 hours. This coupling was then repeated using Boc-β-MeCys(Trt)-OH (50.3 mg, 0.105 mmole; (SEQ ID NO:14)), HOBt (16.1 mg, 0.105 mmole) and DIC (13.2 mg, 0.105 mmole) in NMP for 12 hours. The resulting resin was washed with DMF and DCM.

The resulting protected peptide-resin was deblocked and cleaved with 8% TIS/TFA (1 ml) for 2 hours. The resin was filtered off and washed with TFA (1 ml) and twice with DCM (1 ml). The filtrate was concentrated under nitrogen stream to less than 1 ml, which was poured into cold ether (5 ml). The precipitate formed was centrifuged and collected. The pellet was taken up in water and lyophilized.

This crude product was dissolved in water and purified on a reverse-phase preparative HPLC using a Luna 5μ C₈(2) column (100×20 mm). The column was eluted with a linear gradient from 100% A and 0% B to 70% A and 30% B in 35 minutes, where A was 0.1% TFA in water and B was 0.1% TFA in acetonitrile. The fractions were checked by an analytical HPLC. Those containing pure product were combined and lyophilized to dryness. The purity of the compound was about 99%. 13.9 mg of the final product was obtained. ESI-MS analysis gave the molecular weight at 409.4 (in agreement with the calculated molecular weight of 409.55).

### Example 9

### Preparation of H-Phe-Lys-Gly-S-Ph (SEQ ID NO:17)

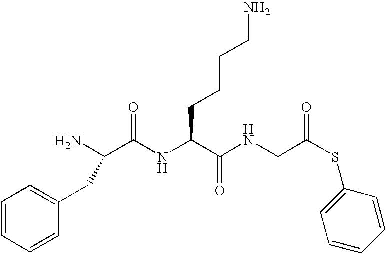

Chlorotrityl chloride resin (1.0 g, 1.49 mmole) (Novabiochem, San Diego, Calif., USA) was treated with a solution of Fmoc-Gly-OH (487 mg, 1.64 mmole; (SEQ ID NO:18)) (Novabiochem, San Diego, Calif., USA) and DIEA (770 mg, 5.96 mmole) in DCM (10 ml) for 1 hour. The resin was filtered and washed with DCM/MeOH/DIEA 17:2:1 (10 ml) twice, with DCM three times, and with DMF three times.

The Fmoc protecting group was removed by shaking the resin with 25% piperidine/DMF (10 ml) for 10 minutes and 30 minutes. The resin was then washed with DMF (10 ml) three times. Fmoc-Lys(Boc)-OH (2.79 g, 5.95 mmole; (SEQ ID NO:15)) (Novabiochem, San Diego, Calif., USA) was coupled to the resulting peptide resin by shaking with HOBt (5.95 mmole) and DIC (5.95 mmole) in NMP (10 ml) for 1 hour.

The deblocking and washing procedures were repeated as above. Boc-Phe-OH (1.58 g, 5.95 mmole; (SEQ ID NO:19)) (Bachem, Torrance, Calif., USA) was coupled to the peptide-resin by shaking with HOBt (5.95 mmole) and DIC (5.95 mmole) in NMP (10 ml) for 1 hour.

The resin was washed with DMF three times, with DCM three times, then with MeOH three times. The resin was dried under vacuum.

The protected peptide was cleaved from the resin by shaking the resin with 10 ml 1.0% TFA in DCM for 2 minutes. The resin was filtered off and the filtrate was drained into 2 ml 10% pyridine in MeOH. After the solvents were removed under vacuum, the residue was taken up in DCM, and washed with saturated NaCl twice and 1 M sodium bisulfate three times. The DCM solution was dried over sodium sulfate. The solvents were removed under vacuum to yield 120 mg of a white solid.

This protected peptide (120 mg, 218 micromole) was treated with TFFH (218 micromole) and DIEA (436 micromole) in DCM (5 ml). The resulting acid fluoride was treated with thiophenol (218 micromole) to form the thioester. After 2 hours, thin layer chromatography (TLC) eluted with 9:1 DCM/MeOH indicated that the reaction was complete. The reaction mixture was diluted with DCM (10 ml) and washed with saturated sodium bicarbonate (5 ml) three times. This solution was dried over sodium sulfate and the solvent was removed under vacuum. The resulting white solid weighed 130 mg.

This protected peptide in 2 ml of DCM was deprotected by addition of 2 ml of TFA. After 2 hours, the solvents were concentrated to 1 ml and the peptide was precipitated by the addition of 14 ml of cold diethyl ether. The resulting suspension was centrifuged and decanted. The pellet was dissolved in water and purified on a reverse-phase preparative HPLC using a Luna 5μ C₈(2) column (100×20 mm). The column was eluted with a linear gradient from 100% A and 0% B to 60% A and 40% B in 30 minutes, where A was 0.1% TFA in water and B was 0.1% TFA in acetonitrile. The fractions were checked by an analytical HPLC. Those containing pure product were combined and lyophilized to dryness. 76.6 mg of the final product was obtained. ESI-MS analysis gave the molecular weight at 442.3 (in agreement with the calculated molecular weight of 442.58).

### Example 10

### Model Ligation using H-β-MeCys-Lys-Phe-NH₂ (SEQ ID NO:13) and H-Phe-Lys-Gly-S-Ph (SEQ ID NO:17)

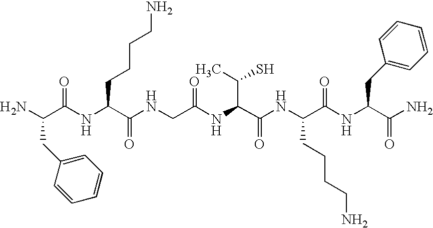

β-MeCys-Lys-Phe-NH₂ (Example 9, 1.0 mg, 2.44 micromole; (SEQ ID NO:13)) was dissolved in 200 mM pH 8.5 phosphate/6 M guanidine buffer (0.1 ml) and tris(carboxyethyl)phosphine (TCEP) (0.035 ml of 40 mg/ml solution, pH adjusted to 7) was added. Phe-Lys-Gly-S-Ph (Example 2, 1.08 mg, 2.44 micromole; (SEQ ID NO:17)) was dissolved in 200 mM pH 8.5 phosphate/6 M guanidine buffer (0.1 ml). The two solutions were combined and the reaction monitored by LC-MS. The ligation was complete at 25 hours.

The resulting solution was purified on reverse phase HPLC (Luna 5μC₈(2) 100×4.6 mm column) eluted from 95% buffer A (0.1% TFA in water) and 5% buffer B (0.1% TFA in acetonitrile) to 20% buffer A and 80% buffer B over 30 minutes monitoring at 220 nm. ESI-MS analysis gave the molecular weight at 741.5 (in agreement with the calculated molecular weight of 741.96).

1. A method for forming an amide bond between a first molecule having a thioester moiety and a second molecule having a β-methyl-cysteine (SEQ ID NO:1) residue having an unoxidized sulfhydryl moiety, comprising the steps of:
(a) reacting said thioester moiety of the first molecule with said unoxidized sulfhydryl moiety of the β-methyl-cysteine (SEQ ID NO:1) residue of the second molecule to generate an intermediate connecting the first and second molecules with a β-amino-thioester linkage; and
(b) allowing the β-amino-thioester linkage of the intermediate to rearrange intramolecularly to from an amide bond connecting said first and second molecules.

2. The method of claim 1, wherein said first and second molecules are independently selected from a group including peptide fragments, polypeptides, peptidomimetics and proteins.

3. The method of claim 1, wherein said reaction and rearrangement steps take place in a solution or a solid phase.

4. The method or claim 1, in which said reaction step takes place in the presence of at least one thiol catalyst.

5. The method of claim 4, wherein said thiol catalyst is selected from the group consisting of thiophenol, 1-thio-2-nitrophenol, 2-thio-benzoic acid, 2-thio-pyridine, 4-thio-2-pyridinecarboxylic acid, 4-thio-2-nitropyridine, 4-mercaptophenylacetic acid, 2-mercaptoethanesulfonic acid, 3-mercapto-1-propanesulfonic acid, and 2,3-dimercaptopropanesulphonic acid.

6. A molecule intermediate, comprising:
(a) a first molecule moiety having a thioester;
(b) a second molecule moiety having a β-methyl-cysteine residue (SEQ ID NO:1); and
(c) a β-amino-thioester linkage connecting the thioester and the β-methyl-cysteine (SEQ ID NO:1).

7. The molecule intermediate of claim 6, wherein said β-amino-thioester linkage spontaneously rearranges intramolecularly to form an amide bond connecting said first and second molecule moieties.

8. A method for synthesizing a polypeptide or a protein by ligation of two peptide fragments, comprising the steps of:
(a) forming an amide bond by ligation of a C-terminal thioester of the first peptide fragment containing N-terminal β-methyl-thiazolidine with an N-terminal β-methyl-cysteine (SEQ ID NO:1) of the second peptide fragment; and
(b) treating the ligation product with a nucleophilic agent under acidic condition to convert the N-terminal β-methyl-thiazolidine residue to the N-terminal β-methyl-cysteine (SEQ ID NO:1) residue.

9. The method as recited in claim 8, wherein said nucleophilic agent is O-alkylhydroxylamine.

10. The method as recited in claim 9, wherein said O-alkylhydroxylamine is O-methylhydroxylamine.

11. The method according to claim 8, wherein said acidic conditions are in the range of pH 2.0 to pH 6.0.

12. The method according to claim 8, wherein said step (a) and step (b) can be repeated until a desired polypeptide or protein is formed.

13. A method for synthesizing a polypeptide containing a free N-terminal β-methyl-cysteine (SEQ ID NO:1), comprising the steps of:
(a) synthesizing a polypeptide containing an N-terminal β-methyl-thiazolidine residue in solid or solution phase; and
(b) (b) treating the polypeptide with a nucleophilic agent under acidic condition to convert the N-terminal β-methyl-thiazolidine residue to a free N-terminal β-methyl-cysteine residue (SEQ ID NO:1).

14. The method as recited in claim 13, wherein said nucleophilic agent is O-alkylhydroxylamine.

15. The method as recited in claim 14, wherein said O-alkylhydroxylamine is O-methylhydroxylamine.

16. A method for synthesizing allo-N-Boc-S-trityl-β-methyl-cysteine (SEQ ID NO:11), comprising the steps of:
(a) converting N-Boc-threonine-t-butylester (SEQ ID NO:4) to N-Boc-O-methanesulfonyl-threonine-t-butylester (SEQ ID NO:6);
(b) converting N-Boc-O-methanesulfonyl-threonine-t-butylester (SEQ ID NO:6) to allo-N-Boc-S-acetyl-β-methyl-cysteine-t-butylester (SEQ ID NO:7);
(c) converting allo-N-Boc-S-acetyl-β-methyl-cysteine-t-butylester (SEQ ID NO:7) to allo-β-methyl-cysteine (SEQ ID NO:8);
(d) converting allo-β-methylcysteine (SEQ ID NO:10) and triphenylmethanol to allo-S-trityl-β-methyl-cysteine (SEQ ID NO:9); and
(e) converting allo-S-trityl-β-methyl-cysteine (SEQ ID NO:9) to allo-N-Boc-S-trityl-β-methyl-cysteine (SEQ ID NO:11).

17. Allo-N-Boc-S-trityl-β-methyl-cysteine (SEQ ID NO:11), synthesized by the method recited in claim 16.

18. A compound of the formula allo-N-Boc-S-trityl-β-methyl-cysteine (SEQ ID NO:11), or a pharmaceutically acceptable salt thereof.

19. A method for synthesizing allo-N-Fmoc-S-trityl-β-methyl-cysteine (SEQ ID NO:12), comprising the steps of:
(a) converting N-Boc-threonine-t-butylester (SEQ ID NO:4) to N-Boc-O-methanesulfonyl-threonine-t-butylester (SEQ ID NO:6);
(b) converting N-Boc-O-methanesulfonyl-threonine-t-butylester (SEQ ID NO:6) to allo-N-Boc-S-acetyl-β-methyl-cysteine-t-butylester (SEQ ID NO:7);
(c) converting allo-N-Boc-S-acetyl-β-methyl-cysteine-t-butylester (SEQ ID NO:7) to allo-β-methyl-cysteine (SEQ ID NO:8);
(d) converting allo-β-methylcysteine (SEQ ID NO:10) and triphenylmethanol to allo-S-trityl-β-methyl-cysteine (SEQ ID NO91); and
(e) converting allo-S-trityl-β-methyl-cysteine (SEQ ID NO:9) to allo-N-Fmoc-S-trityl-β-methyl-cysteine (SEQ ID NO:12).

20. Allo-N-Fmoc-S-trityl-β-methyl-cysteine (SEQ ID NO:12), synthesized by the method recited in claim 19.

21. A compound of the formula allo-N-Fmoc-S-trityl-β-methyl-cysteine (SEQ ID NO:12), or a pharmaceutically acceptable salt thereof.


SEQUENCE LISTING (19 sequences)

SEQ ID NO: 1 - protein, 1 aa, Artificial Sequence
  Intermediate for chemical synthesis of polypeptides and proteins
X

SEQ ID NO: 2 - protein, 1 aa, Artificial Sequence
  Intermediate for chemical synthesis of polypeptides and proteins
X

SEQ ID NO: 3 - protein, 1 aa, Artificial Sequence
  Intermediate for chemical synthesis of polypeptides and proteins
X

SEQ ID NO: 4 - protein, 1 aa, Artificial Sequence
  Intermediate for chemical synthesis of polypeptides and proteins
X

SEQ ID NO: 5 - protein, 1 aa, Artificial Sequence
  Intermediate for chemical synthesis of polypeptides and proteins
X

SEQ ID NO: 6 - protein, 1 aa, Artificial Sequence
  Intermediate for chemical synthesis of polypeptides and proteins
X

SEQ ID NO: 7 - protein, 1 aa, Artificial Sequence
  Intermediate for chemical synthesis of polypeptides and proteins
X

SEQ ID NO: 8 - protein, 1 aa, Artificial Sequence
  Intermediate for chemical synthesis of polypeptides and proteins
X

SEQ ID NO: 9 - protein, 1 aa, Artificial Sequence
  Intermediate for chemical synthesis of polypeptides and proteins
X

SEQ ID NO: 10 - protein, 1 aa, Artificial Sequence
  Intermediate for chemical synthesis of polypeptides and proteins
X

SEQ ID NO: 11 - protein, 1 aa, Artificial Sequence
  Intermediate for chemical synthesis of polypeptides and proteins
X

SEQ ID NO: 12 - protein, 1 aa, Artificial Sequence
  Intermediate for chemical synthesis of polypeptides and proteins
X

SEQ ID NO: 13 - protein, 3 aa, Artificial Sequence
  Intermediate for chemical synthesis of polypeptides and proteins
XKF

SEQ ID NO: 14 - protein, 1 aa, Artificial Sequence
  Intermediate for chemical synthesis of polypeptides and proteins
X

SEQ ID NO: 15 - protein, 1 aa, Artificial Sequence
  Intermediate for chemical synthesis of polypeptides and proteins
X

SEQ ID NO: 16 - protein, 1 aa, Artificial Sequence
  Intermediate for chemical synthesis of polypeptides and proteins
X

SEQ ID NO: 17 - protein, 3 aa, Artificial Sequence
  Intermediate for chemical synthesis of polypeptides and proteins
FKX

SEQ ID NO: 18 - protein, 1 aa, Artificial Sequence
  Intermediate for chemical synthesis of polypeptides and proteins
X

SEQ ID NO: 19 - protein, 1 aa, Artificial Sequence
  Intermediate for chemical synthesis of polypeptides and proteins
X